(19) United States
(12) Patent Application Publication
(10) Pub. No.: US 2008/0138377 A1
(43) Pub. Date: Jun. 12, 2008

(54) Vasodilator Eluting Luminal Stent Devices With A Specific Polyphosphazene Coating and Methods for Their Manufacture and Use

(75) Inventors: Olaf Fritz, Hirschhorn (DE); Ulf Fritz, Hirschhorn (DE); Roman Denk, Weidenstetten (DE); Teresa Wilson, Newnan, GA (US); Ralph E. Gaskins, Atlanta, GA (US)

Correspondence Address: SUTHERLAND ASBILL & BRENNAN LLP, 999 PEACHTREE STREET, N.E., ATLANTA, GA, 30309, US

(73) Assignee: CELONOVA BIOSCIENCES, INC., Newnan, GA (US)

(21) Appl. No.: 11/930,737

(22) Filed: Oct. 31, 2007

Related U.S. Application Data
(63) Continuation-in-part of application No. 11/023,928, filed on Dec. 28, 2004.

(30) Foreign Application Priority Data
Jul. 5, 2002 (DE) 10230190.5
Jul. 4, 2003 (EP) PCT/EP03/07197

(51) Int. Cl. A61K 9/00; A61K 47/16

(52) U.S. Cl. 424/423; 514/788

(57) ABSTRACT

The present invention is directed to luminal stent devices including vascular devices that comprise a specific polyphosphazene and the capability of releasing nitric oxide or other smooth muscle relaxant compounds in vivo or into stored or transient flowing blood to achieve vascular dilatation, reduce adverse reactions, reduce thrombosis, and/or to maintain the patency of a desired anatomic lumen.

### CROSS-REFERENCE TO RELATED APPLICATIONS

This application is a continuation-in-part of U.S. patent application Ser. No. 11/023,928, filed Dec. 28, 2004, which claims the benefit of priority of PCT Patent Application No. PCT/EP03/07197, filed Jul. 4, 2003 and German Patent Application No. DE10230190.5, filed Jul. 5, 2002, the entire disclosures of which are incorporated herein by reference.

### BACKGROUND OF THE INVENTION

The present invention is directed to medical devices including luminal stent devices that comprise a specific polyphosphazene and a capability of releasing nitric oxide or other smooth muscle relaxant compounds in vivo or into stored blood to achieve vascular dilatation, reduce adverse reactions, and reduced thrombosis.

Nitric oxide (NO) is one of the few gaseous biological signaling molecules known. It is a key biological messenger, playing a role in a variety of biological processes. Nitric oxide, also known as the ‘endothelium-derived relaxing factor’, or ‘EDRF’, is biosynthesized from arginine and oxygen by various nitric oxide synthase (NOS) enzymes and by reduction of inorganic nitrate. The endothelial cells that line blood vessels use nitric oxide to signal the surrounding smooth muscle to relax, thus dilating the artery and increasing blood flow. The production of nitric oxide is elevated in populations living at high-altitudes, which helps these people avoid hypoxia. Effects include blood vessel dilatation, and neurotransmission. Nitroglycerin and amyl nitrite serve as vasodilators because they are converted to nitric oxide in the body.

Phosphodiesterase type 5 inhibitors, often shortened to PDE5 inhibitors. are a class of drugs used to block the degradative action of phosphodiesterase type 5 on cyclic GMP in the smooth muscle cells lining blood vessels. NO activates the enzyme guanylate cyclase which results in increased levels of cyclic guanosine monophosphate (cGMP), leading to smooth muscle relaxation in blood vessels. PDE5 inhibitors inhibit the degradation of cGMP by phosphodiesterase type 5 (PDE5).

Nitric oxide is also generated by macrophages and neutrophils as part of the human immune response. Nitric oxide is toxic to bacteria and other human pathogens. In response, however, many bacterial pathogens have evolved mechanisms for nitric oxide resistance.

A biologically important reaction of nitric oxide is S-nitrosylation, the conversion of thiol groups, including cysteine residues in proteins, to form S-nitrosothiols (RSNOs). S-Nitrosylation is a mechanism for dynamic, post-translational regulation of most or all major classes of protein.

Nitroglycerine or glyceryl trinitrate (GTN) has been used to treat angina and heart failure since at least 1880. Despite this, the mechanism of nitric oxide (NO) generation from GTN and the metabolic consequences of this bioactivation are still not entirely understood.

GTN is a pro-drug which must first be denitrated to produce the active metabolite NO. Nitrates which undergo denitration within the body to produce NO are called nitrovasodilators and their denitration occurs via a variety of mechanisms. The mechanism by which nitrates produce NO is widely disputed. Some believe that nitrates produce NO by reacting with sulfhydryl groups, while others believe that enzymes such as glutathione S-transferases, cytochrome P450 (CYP), and xanthine oxidoreductase are the primary source of GTN bioactivation. In recent years a great deal of evidence has been produced which supports the belief that clinically relevant denitration of GIN to produce 1,2-glyceryl dinitrate (GDN) and NO is catalyzed by mitochondrial aldehyde dehydrogenase (mtALDH). NO is a potent activator of guanylyl cyclase (GC) by heme-dependent mechanisms; this activation results in cGMP formation from guanosine triphosphate (GTP). Thus, NO increases the level of cGMP within the cell.

GTP is more useful in preventing angina attacks than reversing them once they have commenced. Patches of glyceryl trinitrate with long activity duration are commercially available. It may also be given as a sublingual dose in the form of a tablet placed under the tongue or a spray into the mouth for the treatment of an angina attack.

Long acting Nitrates can be more useful as they are generally more effective and stable in the short term. GTP is also used to help provoke a vasovagal syncope attack while having a tilt table test which will then give more accurate results.

Vascular stents are widely used in medicine and surgery to counteract significant decreases in vessel or duct diameter by acutely propping open the conduit by mechanical force. Because vascular stents are used to mechanically maintain the patency of blood vessels to maintain or increase blood flow therethrough, they are used to treat the same types of situations as vasodilator drugs, including the nitrites and related agents.

Stents are generally provided as a stent structure of an expandable mesh or framework, which may be fashioned of metal, polymer, or fabric, defining an interior stent lumen. Non-rigid stent structures usually are provided with a rigid or expandable means of supporting the non-rigid tent structure. Stents are typically deployed by expansion of the stent within the targeted anatomic lumen, such as a blood vessel, to maintain a desired patency of the lumen.

Stents are often used to alleviate diminished blood flow to organs and extremities beyond an obstruction in order to maintain an adequate delivery of oxygenated blood. Although the most common use of stents is in coronary arteries, they are widely used to mechanically maintain the patency of anatomic lumens in other natural body conduits, such as central and peripheral arteries and veins, bile ducts, esophagus, colon, trachea or large bronchi, ureters, and urethra. These structures also contain smooth muscle components that could relax as responsive to nitric oxide therapy.

One of the drawbacks of vascular stents is the potential development of a thick smooth muscle tissue inside the lumen, the so-called neointima. Development of a neointima is variable but can at times be so severe as to re-occlude the vessel lumen (restenosis), especially in the ease of smaller diameter vessels, which often results in re-intervention. Consequently, current research focuses on the reduction of neointima after stent placement. Considerable improvements have been made, including the use of more bio-compatible materials, anti-inflammatory drug-eluting stents, resorbable stents, and others. Fortunately, even if stents are eventually covered by neointima, the minimally invasive nature of their deployment makes re-intervention possible and usually straightforward.

It would also be desirable to therapeutically increase the nitrous oxide content in blood in vivo in anatomic areas for treatment for diseases or pathologic conditions in which localized or systemic vasodilatation is compromised.

### BRIEF SUMMARY OF THE INVENTION

The invention includes a coating for luminal stent devices for use in therapeutic settings where it is desirable to have such devices release nitric oxide or other smooth muscle relaxant drugs into blood or into an anatomic space such as a blood vessel, pancreatic duct, bile duct, tear duct, urethra, ureter, esophagus, intestine, penis, or other anatomic structure whose size is controlled by the action of smooth muscle.

The medical devices of the present invention further comprise poly[bis(trifluoroethoxy)phosphazene] and/or a derivative thereof and one or more smooth muscle relaxant active agents. Poly[(bistrifluorethoxy)phosphazene] has antibacterial and anti-inflammatory properties and inhibits the accumulation of thrombocytes.

Further described herein is a method of delivering an active agent capable of eluting nitric oxide or other smooth muscle relaxants from within a specific polyphosphazene coating into an anatomic area or a container space is therapeutically desirable.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments that are presently preferred. It should be understood, however, that the invention is not limited to the precise arrangements and instrumentalities shown.

In the drawings:

FIG. 1A shows a surface of a strut of a vascular sent of the present invention.

FIG. 1B shows a cross section of a strut of a vascular sent of the present invention at the points A-A′ on FIG. 1.

### DETAILED DESCRIPTION OF THE INVENTION

The present invention may be understood more readily by reference to the following detailed description of the preferred embodiments of the invention and the examples included herein. However, before the preferred embodiments of the devices and methods according to the present invention are disclosed and described, it is to be understood that this invention is not limited to the exemplary embodiments described within this disclosure, and the numerous modifications and variations therein that will be apparent to those skilled in the art remain within the scope of the invention disclosed herein. It is also to be understood that the terminology used herein is for the purpose of describing specific embodiments only and is not intended to be limiting.

Unless otherwise noted, the terms used herein are to be understood according to conventional usage by those of ordinary skill in the relevant art. In addition to the definitions of terms provided below, it is to be understood that as used in the specification and in the claims, “a” or “an” can mean one or more, depending upon the context in which it is used.

Described herein are luminal stent devices comprising poly[bis(trifluoroethoxy)phosphazene] and/or a derivative thereof and one or smooth muscle relaxant active agents capable of in vivo release into the tissues or organs of a mammalian patient upon implantation, deployment, or use of the devices to maintain patency of a desired anatomic lumen.

Further described herein are methods for the manufacture and use of medical devices comprising poly[bis(trifluoroethoxy)phosphazene] and/or a derivative thereof and one or more nitrogen compounds or other smooth muscle relaxant active agents capable of release during storage of biological or pharmaceutical containment or administration therein, or in vivo release into the tissues or organs of a mammalian patient upon implantation, deployment, or use of the devices to maintain patency of a desired anatomic lumen.

In certain embodiments of the present invention, medical devices are provided with a polymeric coating comprising poly[bis(trifluoroethoxy)phosphazene] and/or a derivative thereof releasably bonded to compounds capable of producing nitric oxide or other bioactive nitrogen compounds upon release in vivo from the polymer.

The present invention further includes methods for the manufacture and use of medical devices comprising a polymeric coating comprising poly[bis(trifluoroethoxy)phosphazene] and/or a derivative thereof releasably bonded to compounds capable of producing nitric oxide or other bioactive nitrogen compounds upon release from the polymer.

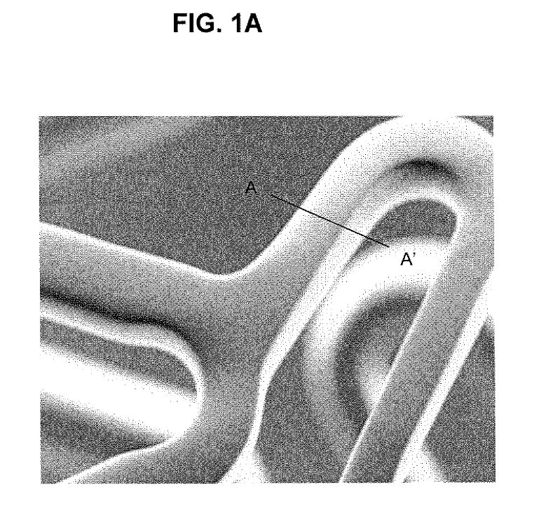

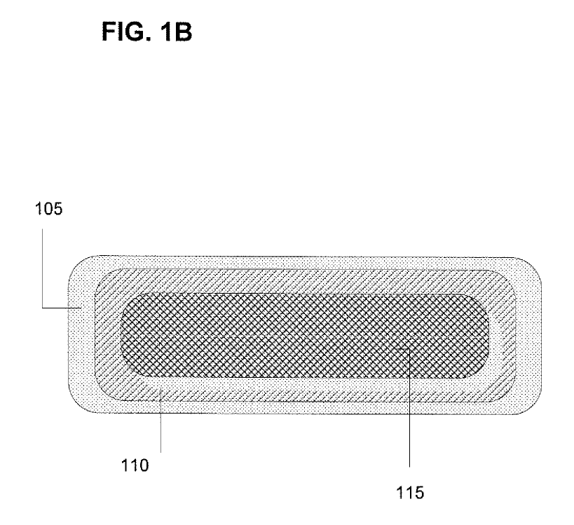

Referring now to FIG. 1A, a detail is shown of a vascular stent comprising a plurality of struts. A cross-sectional drawing of an exemplary strut of a vascular stent according to the present invention is shown in FIG. 1B.

In FIG. 1B, a stent structure 115 is coated with an adherent subcoating of a nitrite compound 110, which is covalently bonded to an exterior coating 105 comprising a polymer poly[bis(2,2,2-trifluoroethoxy)phosphazene] or a derivative thereof (referred to further herein as “poly[bis(trifluoroethoxyphosphazene]”.

The nitrite subcoating 110 as shown in FIG. 1B may be any nitrogen compound capable of in vivo breakdown to nitric oxide or other smooth muscle relaxant nitrite or nitrate compounds. In alternate embodiments of the present invention, the subcoating may be a non-nitrogen based smooth muscle relaxant agent. In the exemplary FIG. 1B section, the nitrite subcoating 110 is shown as a separate layer, adherent to the substrate of stent structure 115 and covalently bonded or otherwise adherent to the polymeric coating 105. In still other embodiments of the present invention, the smooth muscle relaxant agent may be integrated into the polymeric coating 105.

As described herein, the polymer poly[bis(2,2,2-trifluoroethoxy)phosphazene] or derivatives thereof have chemical and biological qualities that distinguish this polymer from other know polymers in general, and from other know polyphosphazenes in particular. In one aspect of this invention, the polyphosphazene is poly[bis(2,2,2-trifluoroethoxy)phosphazene] or derivatives thereof such as other alkoxide, halogenated alkoxide, or fluorinated alkoxide substituted analogs thereof. The preferred poly[bis(trifluoroethoxy)phosphazene] polymer is made up of repeating monomers represented by the formula (I) shown below:

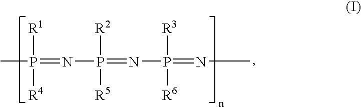

wherein R¹ to R⁶ are all trifluoroethoxy (OCH₂CF₃) groups, and wherein n may vary from at least about 40 to about 100,000′ as disclosed herein. Alternatively, one may use derivatives of this polymer in the present invention. The term “derivative” or “derivatives” is meant to refer to polymers made up of monomers having the structure of formula I but where one or more of the R¹ to R⁶ functional group(s) is replaced by a different functional group(s), such as an unsubstituted alkoxide, a halogenated alkoxide, a fluorinated alkoxide, or any combination thereof, or where one or more of the R¹ to R⁶ is replaced by any of the other functional group(s) disclosed herein, but where the biological inertness of the polymer is not substantially altered.

In one aspect of the polyphosphazene of formula (I) illustrated above, for example, at least one of the substituents R¹ to R⁶ can be an unsubstituted alkoxy substituent, such as methoxy (OCH₃)₃, ethoxy (OCH₂CH₃) or n-propoxy (OCH₂CH₂CH₃). In another aspect, for example, at least one of the substituents R¹ to R⁶ is an alkoxy group substituted with at least one fluorine atom. Examples of useful fluorine-substituted alkoxy groups R¹ to R⁶ include, but are not limited to OCF₃, OCH₂CF₃, OCH₂CH₂CF₃, OCH₂CF₂CF₃, OCH(CF₃)₂, OCCH₃(CF₃)₂, OCH₂CF₂CF₂CF₃, OCH₂(CF₂)₃CF₃, OCH₂(CF₂)₄CF₃, OCH₂(CF₂)₅CF₃, OCH₂(CF₂)₆CF₃, OCH₂(CF₂)₇CF₃, OCH₂CF₂CHF₂, OCH₂CF₂CF₂CHF₂, OCH₂(CF₂)₃CHF₂, OCH₂(CF₂)₄CHF₂, OCH₂(CF₂)₅CHF₂, OCH₂(CF₂)₆CHF₂, OCH₂(CF₂)₇CHF₂, and the like. Thus, while trifluoroethoxy (OCH₂CF₃) groups are preferred, these further exemplary functional groups also may be used alone, in combination with trifluoroethoxy, or in combination with each other. In one aspect, examples of especially useful fluorinated alkoxide functional groups that may be used include, but are not limited to 2,2,3,3,3-pentafluoropropyloxy (OCH₂CF₂CF₃), 2,2,2,2′,2′,2′-hexafluoroisopropyloxy (OCH(CF₃)₂), 2,2,3,3,4,4,4-heptafluorobutyloxy (OCH₂CF₂CF₂CF₃), 3,3,4,4,5,5,6,6,7,7,8,8,8-tridecafluorooctyloxy (OCH₂(CF₂)₇CF₃), 2,2,3,3,-tetrafluoropropyloxy (OCH₂CF₂CHF₂), 2,2,3,3,4,4-hexafluorobutyloxy (OCH₂CF₂CF₂CHF₂), 3,3,4,4,5,5,6,6,7,7,8,8-dodecafluorooctyloxy (OCH₂(CF₂)₇CHF₂), and the like, including combinations thereof.

Further, in some embodiments, 1% or less of the R¹ to R⁶ groups may be alkenoxy groups, a feature that may assist in crosslinking to provide a more elastomeric phosphazene polymer. In this aspect, alkenoxy groups include, but are not limited to, OCH₂CH═CH₂, OCH₂CH₂CH═CH₂, allylphenoxy groups, and the like, including combinations thereof. Also in formula (I) illustrated herein, the residues R¹ to R⁶ are each independently variable and therefore can be the same or different.

By indicating that n can be as large as ∞ in formula I, it is intended to specify values of n that encompass polyphosphazene polymers that can have an average molecular weight of up to about 75 million Daltons. For example, in one aspect, n can vary from at least about 40 to about 100,000. In another aspect, by indicating that n can be as large as ∞ in formula I, it is intended to specify values of n from about 4,000 to about 50,000, more preferably, n is about 7,000 to about 40,000 and most preferably n is about 13,000 to about 30,000.

In another aspect of this invention, the polymer used to prepare the polymers disclosed herein has a molecular weight based on the above formula, which can be a molecular weight of at least about 70,000 g/mol, more preferably at least about 1,000,000 g/mol, and still more preferably a molecular weight of at least about 3×10⁶ g/mol to about 20×10⁶ g/mol. Most preferred are polymers having molecular weights of at least about 10,000,000 g/mol.

In a further aspect of the polyphosphazene formula (I) illustrated herein, n is 2 to ∞, and R¹ to R⁶ are groups which are each selected independently from alkyl, aminoalkyl, haloalkyl, thioalkyl, thioaryl, alkoxy, haloalkoxy, aryloxy, haloaryloxy, alkylthiolate, arylthiolate, alkylsulphonyl, alkylamino, dialkylamino, heterocycloalkyl comprising one or more heteroatoms selected from nitrogen, oxygen, sulfur, phosphorus, or a combination thereof or heteroaryl comprising one or more heteroatoms selected from nitrogen, oxygen, sulfur, phosphorus, or a combination thereof. In this aspect of formula (I), the pendant side groups or moieties (also termed “residues”) R¹ to R⁶ are each independently variable and therefore can be the same or different. Further, R¹ to R⁶ can be substituted or unsubstituted. The alkyl groups or moieties within the alkoxy, alkylsulphonyl, dialkylamino, and other alkyl-containing groups can be, for example, straight or branched chain alkyl groups having from 1 to 20 carbon atoms, typically from 1 to 12 carbon atoms, it being possible for the alkyl groups to be further substituted, for example, by at least one halogen atom, such as a fluorine atom or other functional group such as those noted for the R¹ to R⁶ groups above. By specifying alkyl groups such as propyl or butyl, it is intended to encompass any isomer of the particular alkyl group.

In one aspect, examples of alkoxy groups include, but are not limited to, methoxy, ethoxy, propoxy, and butoxy groups, and the like, which can also be further substituted. For example the alkoxy group can be substituted by at least one fluorine atom, with 2,2,2-trifluoroethoxy constituting a useful alkoxy group. In another aspect, one or more of the alkoxy groups contains at least one fluorine atom. Further, the alkoxy group can contain at least two fluorine atoms or the alkoxy group can contain three fluorine atoms. For example, the polyphosphazene that is combined with the silicone can be poly[bis(2,2,2-trifluoroethoxy)phosphazene]. Alkoxy groups of the polymer can also be combinations of the aforementioned embodiments wherein one or more fluorine atoms are present on the polyphosphazene in combination with other groups or atoms.

Examples of alkylsulphonyl substituents include, but are not limited to, methylsulphonyl, ethylsulphonyl, propylsulphonyl, and butylsulphonyl groups. Examples of dialkylamino substituents include, but are not limited to, dimethyl-, diethyl-, dipropyl-, and dibutylamino groups. Again, by specifying alkyl groups such as propyl or butyl, it is intended to encompass any isomer of the particular alkyl group.

Exemplary aryloxy groups include, for example, compounds having one or more aromatic ring systems having at least one oxygen atom, non-oxygenated atom, and/or rings having alkoxy substituents, it being possible for the aryl group to be substituted for example by at least one alkyl or alkoxy substituent defined above. Examples of aryloxy groups include, but are not limited to, phenoxy and naphthoxy groups, and derivatives thereof including, for example, substituted phenoxy and naphthoxy groups.

The heterocycloalkyl group can be, for example, a ring system which contains from 3 to 10 atoms, at least one ring atom being a nitrogen, oxygen, sulfur, phosphorus, or any combination of these heteroatoms. The hetereocycloalkyl group can be substituted, for example, by at least one alkyl or alkoxy substituent as defined above. Examples of heterocycloalkyl groups include, but are not limited to, piperidinyl, piperazinyl, pyrrolidinyl, and morpholinyl groups, and substituted analogs thereof.

The heteroaryl group can be, for example, a compound having one or more aromatic ring systems, at least one ring atom being a nitrogen, an oxygen, a sulfur, a phosphorus, or any combination of these heteroatoms. The heteroaryl group can be substituted for example by at least one alkyl or alkoxy substituent defined above. Examples of heteroaryl groups include, but are not limited to, imidazolyl, thiophene, furane, oxazolyl, pyrrolyl, pyridinyl, pyridinoyl, isoquinolinyl, and quinolinyl groups, and derivatives thereof such as substituted groups.

As disclosed herein, smooth muscle relaxant active agents or compounds capable of producing nitric oxide or other bioactive nitrogen compounds in vivo upon release from the present invention further comprise diazeniumdiolates, sodium nitroprusside, molsidomine, nitrate esters, the S-nitrosothiol family, L-arginine, nitric oxide-nucleophile complexes, glyceryl trinitrate, nitric oxide-primary amine complexes, and related compounds, esters, amines, or other compositions thereof. Smooth muscle relaxant active agents or compounds capable of producing nitric oxide or other bioactive nitrogen compounds upon release of the present invention may further comprise any other inorganic or organic composition capable of forming nitric oxide upon chemical degradation.

In certain preferred embodiments of the present invention, diazeniumdiolates are incorporated into blood-insoluble polyphosphazene polymers that generate molecular NO at their surfaces. In other preferred embodiments of the present invention, diazeniumdiolates may be applied to a substrate surface of a medical device as an intermediate coating, which is then coated with the preferred poly[bis(trifluoroethoxy)phosphazene] polymer of the present invention. In yet other preferred embodiments of the present invention, a substrate surface of a medical device may receive a first coating with the preferred poly[bis(trifluoroethoxy)phosphazene] polymer of the present invention, followed by an intermediate coating of diazeniumdiolates, followed by a second coating of the poly[bis(trifluoroethoxy)phosphazene] polymer as described herein. In such embodiments with a first and second coating of the poly[bis(trifluoroethoxy)phosphazene] polymer, the first and second coatings may each be bioabsorbable or non-bioabsorbable.

Diazeniumdiolates are now available with a range of half-lives for spontaneous NO release. The ability of the diazeniumdiolates to generate copious NO at rates that vary widely is largely independent of metabolic or medium effects.

Other preferred embodiments of the present invention may use other nitric oxide-eluting or other smooth muscle relaxant compounds, including, but not limited to sodium nitroprusside, molsidomine, nitrate esters, the S-nitrosothiol family, L-arginine, nitric oxide-nucleophile complexes, glyceryl trinitrate, nitric oxide-primary amine complexes, and related compounds. In such various embodiments of the present invention, the nitric oxide-eluting or other smooth muscle relaxant compounds may be incorporated into non-bioabsorbable polyphosphazene polymers that generate molecular NO at their surfaces. In other preferred embodiments of the present invention, nitric oxide-eluting or other smooth muscle relaxant compounds may be applied to a substrate surface of a medical device as an intermediate coating, which is then coated with the preferred poly[bis(trifluoroethoxy)phosphazene] polymer of the present invention. In yet other preferred embodiments of the present invention, a substrate surface of a medical device may receive a first coating with the preferred poly[bis(trifluoroethoxy)phosphazene] polymer of the present invention, followed by an intermediate coating of nitric oxide-eluting or other smooth muscle relaxant compounds, followed by a second coating of the poly[bis(trifluoroethoxy)phosphazene] polymer as described herein. In such embodiments with a first and second coating of the poly[bis(trifluoroethoxy)phosphazene] polymer, the first and second coatings may each be bioabsorbable or non-bioabsorbable.

The medical devices disclosed herein may comprise the poly[bis(trifluoroethoxy)phosphazene] polymer represented by formula (I) in various forms: as a coating, as a film, or as a solid structural component. When used as a coating or film in embodiments of the present invention, the poly[bis(trifluoroethoxy)phosphazene] polymer may be provided in varying degrees of porosity, or as a solid surface. Coatings of medical devices of the present invention may be accomplished by any known coating process, including but not limited to dip coating, spray coating, spin coating, brush coating, electrostatic coating, electroplating, electron beam-physical vapor deposition, and other coating technologies.

Similarly, the poly[bis(trifluoroethoxy)phosphazene] polymer may be provided as either a bioabsorbable or non-bioabsorbable form as most appropriate in various embodiments of the present invention. In various embodiments of the present invention, two or more coatings of the poly[bis(trifluoroethoxy)phosphazene] polymer may be applied to the surface of a medical device, and the two or more coatings of the poly[bis(trifluoroethoxy)phosphazene] polymer may be independently provided as bioabsorbable or non-bioabsorbable.

In one embodiment of the present invention an adhesion promoter may be provided in a layer between the surface of the substrate and the polymeric coating.

In exemplary embodiments of the present invention, the adhesion promoter is an organosilicon compound, preferably an amino-terminated silane or a compound based on an aminosilane, or an alkylphosphonic acid Aminopropyltrimethoxysilane is a preferred adhesion promoter according to the present invention.

In various exemplary embodiments of the present invention, the adhesion promoter particularly improves the adhesion of the coating to the surface of the implant material through coupling of the adhesion promoter to the surface of the implant material, through, for instance, ionic and/or covalent bonds, and through further coupling of the adhesion promoter to reactive components, particularly to the antithrombogenic polymer of the coating, through, for instance, ionic and/or covalent bonds.

It will be appreciated by those possessing ordinary skill in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A luminal stent device, comprising:
a. an expandable stent structure for placement in a desired anatomic lumen to maintain patency therewithin;
b. a specific polyphosphazene component, the polyphosphazene having the formula: 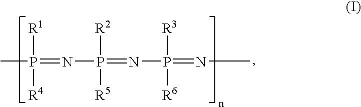
n is 2 to ∞; and
R¹ to R⁶ are each selected independently from alkyl, aminoalkyl, haloalkyl, thioalkyl, thioaryl, alkoxy, haloalkoxy, aryloxy, haloaryloxy, alkylthiolate, arylthiolate, alkylsulphonyl, alkylamino, dialkylamino, heterocycloalkyl comprising one or more heteroatoms selected from nitrogen, oxygen, sulfur, phosphorus, or a combination thereof, or heteroaryl comprising one or more heteroatoms selected from nitrogen, oxygen, sulfur, phosphorus, or a combination thereof.
c. a smooth muscle relaxant active agent.

2. The luminal stent device according to claim 1, wherein at least one of R¹ to R⁶ is an alkoxy group substituted with at least one fluorine atom.

3. The luminal stent device according to claim 1, wherein R¹ to R⁶ are selected independently from OCH₃, OCH₂CH₃, OCH₂CH₂CH₃, OCF₃, OCH₂CF₃, OCH₂CH₂CF₃, OCH₂CF₂CF₃, OCH(CF₃)₂, OCCH₃(CF₃)₂, OCH₂CF₂CF₂CF₃, OCH₂(CF₂)₃CF₃, OCH₂(CF₂)₄CF₃, OCH₂(CF₂)₅CF₃, OCH₂(CF₂)₆CF₃, OCH₂(CF₂)₇CF₃, OCH₂CF₂CHF₂, OCH₂CF₂CF₂CHF₂, OCH₂(CF₂)₃CHF₂, OCH₂(CF₂)₄CHF₂, OCH₂(CF₂)₅CHF₂ OCH₂(CF₂)CHF₂, OCH₂(CF₂)₇CHF₂, OCH₂CH═CH₂, OCH₂CH₂CH═CH₂, or any combination thereof.

4. The luminal stent device according to claim 1, wherein the polyphosphazene is poly[bis(2,2,2-trifluoroethoxy)]phosphazene or a derivative of poly[bis(2,2,2-trifluoroethoxy)]phosphazene.

5. The luminal stent device according to claim 1, wherein the polyphosphazene component is a coating for the expandable stent structure.

6. The luminal stent device according to claim 1, wherein the smooth muscle relaxant active agent is releasably bonded to the polyphosphazene component.

7. The luminal stent device according to claim 1, wherein the smooth muscle relaxant active agent is a compound capable of producing nitric oxide or other bioactive nitrogen compounds upon in vivo release in the desired anatomic lumen.

8. The luminal stent device according to claim 1, wherein the anatomic lumen is a vascular lumen.

9. The luminal stent device according to claim 1, wherein the anatomic lumen is a pancreatic duct, bile duct, tear duct, urethra, ureter, esophagus, or intestine.

10. A coating for a luminal stent device, comprising:
a. a specific polyphosphazene coating, the polyphosphazene having the formula: 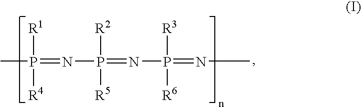
n is 2 to ∞; and
R¹ to R⁶ are each selected independently from alkyl, aminoalkyl, haloalkyl, thioalkyl, thioaryl, alkoxy, haloalkoxy, aryloxy, haloaryloxy, alkylthiolate, arylthiolate, alkylsulphonyl, alkylamino, dialkylamino, heterocycloalkyl comprising one or more heteroatoms selected from nitrogen, oxygen, sulfur, phosphorus, or a combination thereof, or heteroaryl comprising one or more heteroatoms selected from nitrogen, oxygen, sulfur, phosphorus, or a combination thereof.
b. a smooth muscle relaxant active agent.

11. The coating according to claim 10, wherein at least one of R¹ to R⁶ is an alkoxy group substituted with at least one fluorine atom.

12. The coating according to claim 10, wherein R¹ to R⁶ are selected independently from OCH₃, OCH₂CH₃, OCH₂CH₂CH₃, OCF₃, OCH₂CF₃, OCH₂CH₂CF₃, OCH₂CF₂CF₃, OCH(CF₃)₂, OCCH₃(CF₃)₂, OCH₂CF₂CF₂CF₃, OCH₂(CF₂)₃CF₃, OCH₂(CF₂)₄CF₃, OCH₂(CF₂)₅CF₃, OCH₂(CF₂)₆CF₃, OCH₂(CF₂)₇CF₃, OCH₂CF₂CHF₂, OCH₂CF₂CF₂CHF₂, OCH₂(CF₂)₃CHF₂, OCH₂(CF₂)₄CHF₂, OCH₂(CF₂)₅CHF₂, OCH₂(CF₂)₆CHF₂, OCH₂(CF₂)₇CHF₂, OCH₂CH═CH₂, OCH₂CH₂CH═CH₂, or any combination thereof.

13. The coating according to claim 10, wherein the polyphosphazene is poly[bis(2,2,2-trifluoroethoxy)]phosphazene or a derivative of poly[bis(2,2,2-trifluoroethoxy)]phosphazene.

14. The coating according to claim 10, wherein the polyphosphazene component is a coating for the expandable stent structure.

15. The coating according to claim 10, wherein the smooth muscle relaxant active agent is releasably bonded to the polyphosphazene component.

16. The coating according to claim 10, wherein the smooth muscle relaxant active agent is a compound capable of producing nitric oxide or other bioactive nitrogen compounds upon in vivo release.

17. The coating according to claim 10, wherein the desired anatomic lumen is a vascular lumen.

18. The coating according to claim 10, wherein the desired anatomic lumen is a pancreatic duct, bile duct, tear duct, urethra, ureter, esophagus, or intestine.

19. The coating according to claim 10, wherein the coating is applied to a substrate surface of a luminal stent device by dip coating, spray coating, spin coating, brush coating, electrostatic coating, electroplating, or electron beam-physical vapor deposition.

20. A method of maintaining patency of a desired anatomic lumen, comprising:
a. selecting a desired anatomic lumen;
b. providing a luminal stent device comprising
(i) an expandable stent structure for placement in a desired anatomic lumen to maintain patency therewithin;
(ii) a specific polyphosphazene component, the polyphosphazene having the formula: 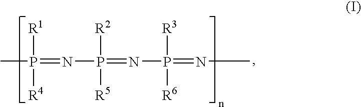
where n is 2 to ∞; and
R¹ to R⁶ are each selected independently from alkyl, aminoalkyl, haloalkyl, thioalkyl, thioaryl, alkoxy, haloalkoxy, aryloxy, haloaryloxy, alkylthiolate, arylthiolate, alkylsulphonyl, alkylamino, dialkylamino, heterocycloalkyl comprising one or more heteroatoms selected from nitrogen, oxygen, sulfur, phosphorus, or a combination thereof, or heteroaryl comprising one or more heteroatoms selected from nitrogen, oxygen, sulfur, phosphorus, or a combination thereof; and
(iii) a smooth muscle relaxant active agent;
c. inserting the device into the desired anatomic lumen;
d. expanding the expandable stent structure to a desired diameter; and
e. releasing the smooth muscle relaxant active agent.

21. The method according to claim 20, wherein the polyphosphazene is poly[bis(2,2,2-trifluoroethoxy)]phosphazene or a derivative of poly[bis(2,2,2-trifluoroethoxy)]phosphazene.

22. The method according to claim 20, wherein the polyphosphazene component is a coating for the expandable stent structure.

23. The method according to claim 20, wherein the smooth muscle relaxant active agent is releasably bonded to the polyphosphazene component.

24. The method according to claim 20, wherein the smooth muscle relaxant active agent is a compound capable of producing nitric oxide or other bioactive nitrogen compounds upon in vivo release.

25. The method according to claim 20, wherein the desired anatomic lumen is a vascular lumen, pancreatic duct, bile duct, tear duct, urethra, ureter, esophagus, or intestine.
